(19) United States
(12) Patent Application Publication
(10) Pub. No.: US 2023/0302071 A1
(43) Pub. Date: Sep. 28, 2023

(54) ANTIDEPRESSANT COMPOSITION CONTAINING PLEUROTUS ERYNGII EXTRACT AS ACTIVE INGREDIENT

(71) Applicant: KOREA UNIVERSITY RESEARCH AND BUSINESS FOUNDATION, Seoul (KR)

(72) Inventor: Cheol Won YUN, Seoul (KR)

(73) Assignee: KOREA UNIVERSITY RESEARCH AND BUSINESS FOUNDATION, Seoul (KR)

(21) Appl. No.: 18/040,535

(22) PCT Filed: Aug. 4, 2021
(86) PCT No.: PCT/KR2021/010219

§ 371 Date: Feb. 3, 2023

(30) Foreign Application Priority Data
Aug. 4, 2020 (KR) 10-2020-0097287
Oct. 20, 2020 (KR) 10-2020-0136185

(51) Int. Cl. A61K 36/07; A23L 33/10

(52) U.S. Cl. CPC A61K 36/07; A23L 33/10; A61K 2236/51; A61K 2236/33

(57) ABSTRACT

Disclosed is an antidepressant composition containing a Pleurotus eryngii extract as an active ingredient, and particularly a food composition and a pharmaceutical composition for preventing, ameliorating, or treating depression containing an extract or fraction of Pleurotus eryngii as an active ingredient. The Pleurotus eryngii extract can inhibit binding between serotonin receptors and selective serotonin reuptake inhibitors by acting on serotonin receptors, can activate signaling mediated by serotonin receptors by acting on serotonin receptors, and can reduce immobility time in a forced swimming test using animal models, and is thus useful in functional foods and pharmaceuticals for preventing, ameliorating, or treating depression.

### TECHNICAL FIELD

The present invention relates to an antidepressant composition containing a Pleurotus eryngii extract as an active ingredient.

### BACKGROUND ART

Depression is a severe and recurrent mental disorder that causes significant disability and financial costs worldwide (Arai et al., 2012; Lucca et al., 2009; Yi et al., 2013). The World Health Organization (WHO) predicts that depression will become the second largest cause of human disability-adjusted life years and has also announced that depression is a cause of physical disability and high suicide rates.

Antidepressants that are currently used are drugs that mainly affect the serotonergic, noradrenergic, and dopaminergic systems, and representative drugs for use in treatment of depressive disorders include tricyclic antidepressants (TCAs), monoamine oxidase inhibitors (MAOIs), selective serotonin reuptake inhibitors (SSRIs), serotonin and noradrenaline reuptake inhibitors (SNRIs), and noradrenaline and dopamine reuptake inhibitors (NDRIs).

Over the past few years, SSRIs and SNRIs have been replaced by TCAs as selective drugs for treatment of depressive disorders in the general public, primarily because of improved tolerability and safety profiles thereof.

However, most antidepressants currently used for treatment are artificially manufactured drugs composed of synthetic compounds, and have side effects such as sexual dysfunction and weight gain, which is undesirable.

Hence, there is a need to develop a new therapeutic agent using a natural material that has superior effects on preventing, ameliorating, or treating depression without side effects.

Meanwhile, Pleurotus eryngii is a type of mushroom widely used as a food ingredient and traditional medicine in Korea. Pleurotus eryngii contains almost no fat or carbohydrates, but has high protein content compared to most vegetables. It is also rich in vitamins B, D, K, A, and C, and is known as a food material suitable for low-calorie meals. Moreover, it is known that Pleurotus eryngii has antimicrobial, antioxidant, anti-inflammatory, and anti-allergic activities in relation to useful physiological activities, but no study has been reported on the relevance thereof to antidepressant activity.

### DISCLOSURE

### Technical Problem

Accordingly, the present inventors have made great efforts to develop new antidepressants using natural materials and ascertained that fractions obtained from Pleurotus eryngii have superior antidepressant activity, thus culminating in the present invention.

Therefore, it is an object of the present invention to provide a food composition for preventing or ameliorating depression containing an extract or fraction of Pleurotus eryngii as an active ingredient.

It is another object of the present invention to provide a pharmaceutical composition for preventing or treating depression containing an extract or fraction of Pleurotus eryngii as an active ingredient.

It is still another object of the present invention to provide a method of preparing a fraction of a Pleurotus eryngii ethanol extract having antidepressant activity.

### Technical Solution

In order to accomplish the above objects, the present invention provides a food composition for preventing or ameliorating depression containing an extract or fraction of Pleurotus eryngii as an active ingredient.

In an embodiment of the present invention, the extract of Pleurotus eryngii may be an ethanol extract of Pleurotus eryngii obtained by adding ethanol to Pleurotus eryngii.

In an embodiment of the present invention, the fraction of Pleurotus eryngii may be a primary ethanol fraction obtained in a manner in which the ethanol extract of Pleurotus eryngii is dried to remove ethanol therefrom, dissolved in water, loaded onto an XCD-20 column, and eluted using ethanol as a solvent; or a secondary methanol fraction obtained in a manner in which the primary ethanol fraction is dried, dissolved in water, loaded onto an HPLC column, and eluted using a concentration gradient of water and methanol.

In an embodiment of the present invention, the extract or fraction of Pleurotus eryngii may inhibit binding between a serotonin reuptake inhibitor and a serotonin receptor, and may activate signaling mediated by a serotonin receptor by binding to the serotonin receptor.

In an embodiment of the present invention, the Pleurotus eryngii may be a Pleurotus eryngii powder obtained by subjecting Pleurotus eryngii to chopping, drying at a temperature of 50 to 70° C. for 10 to 14 hours, and then grinding.

In addition, the present invention provides a pharmaceutical composition for preventing or treating depression containing an extract or fraction of Pleurotus eryngii as an active ingredient.

In an embodiment of the present invention, the extract of Pleurotus eryngii may be an ethanol extract of Pleurotus eryngii obtained by adding ethanol to Pleurotus eryngii.

In an embodiment of the present invention, the fraction of Pleurotus eryngii may be a primary ethanol fraction obtained in a manner in which the ethanol extract of Pleurotus eryngii is dried to remove ethanol therefrom, dissolved in water, loaded onto an XCD-20 column, and eluted using ethanol as a solvent; or a secondary methanol fraction obtained in a manner in which the primary ethanol fraction is dried, dissolved in water, loaded onto an HPLC column, and eluted using a concentration gradient of water and methanol.

In an embodiment of the present invention, the extract or fraction of Pleurotus eryngii may inhibit binding between a serotonin reuptake inhibitor and a serotonin receptor, and may activate signaling mediated by a serotonin receptor by binding to the serotonin receptor.

In an embodiment of the present invention, the Pleurotus eryngii may be a Pleurotus eryngii powder obtained by subjecting Pleurotus eryngii to chopping, drying at a temperature of 50 to 70° C. for 10 to 14 hours, and then grinding.

In addition, the present invention provides a method of preparing a fraction of a Pleurotus eryngii ethanol extract having antidepressant activity, including: (1) preparing a Pleurotus eryngii powder by subjecting Pleurotus eryngii to chopping, drying at a temperature of 50 to 70° C. for 10 to 14 hours, and then grinding; (2) obtaining an ethanol extract of Pleurotus eryngii by adding ethanol to the Pleurotus eryngii powder and performing extraction for 20 to 24 hours; (3) obtaining a primary ethanol fraction by subjecting the ethanol extract of Pleurotus eryngii to drying to remove ethanol therefrom, dissolution in water, loading onto an XCD-20 column, and elution using ethanol as a solvent; and (4) obtaining a secondary methanol fraction by subjecting the primary ethanol fraction to drying, dissolution in water, loading onto an HPLC column, and elution using a concentration gradient of water and methanol.

### Advantageous Effects

According to the present invention, a Pleurotus eryngii extract can inhibit binding between serotonin receptors and selective serotonin reuptake inhibitors by acting on serotonin receptors, can activate signaling mediated by serotonin receptors by acting on serotonin receptors, and can reduce immobility time in a forced swimming test using animal models, and is thus useful in functional foods and pharmaceuticals for preventing, ameliorating, or treating depression.

### DESCRIPTION OF DRAWINGS

FIG. 1 shows results of obtaining the extract and fractions having antidepressant activity from Pleurotus eryngii;

FIG. 2 shows results of analyzing the serotonin binding inhibitory response when using the fractions of the Pleurotus eryngii extract according to the present invention;

FIG. 3 shows results of analyzing the serotonin binding inhibitory response in an AGS cell line when using the fractions of the Pleurotus eryngii extract according to the present invention;

FIG. 4 shows results confirming the activation of serotonin receptor-mediated signaling in the AGS cell line when using the fractions of the Pleurotus eryngii extract according to the present invention; and

FIG. 5 shows results of analyzing the antidepressant efficacy (reduction in immobility time) of the Pleurotus eryngii extract and fractions according to the present invention in a forced swimming test using animal models.

### BEST MODE

The present invention pertains to an antidepressant composition containing a Pleurotus eryngii extract as an active ingredient.

The present inventors have studied new natural materials having preventive, ameliorative, or therapeutic activity on depression, and ascertained that a Pleurotus eryngii extract and fractions thereof have superior antidepressant activity.

In an embodiment of the present invention, the antidepressant activity of an ethanol extract of Pleurotus eryngii obtained by adding ethanol to Pleurotus eryngii and fractions obtained by purifying the ethanol extract through column chromatography was analyzed.

Specifically, the binding inhibitory activity between serotonin receptors and serotonin reuptake inhibitors was analyzed using the Pleurotus eryngii extract and fractions according to the present invention, confirming that the Pleurotus eryngii extract and fractions according to the present invention were capable of effectively inhibiting binding between serotonin receptors and serotonin reuptake inhibitors, and also that the Pleurotus eryngii extract and fractions according to the present invention activated signaling mediated by serotonin receptors by binding to serotonin receptors.

In another embodiment of the present invention, the Pleurotus eryngii extract and fractions according to the present invention were confirmed to reduce the immobility time of mice upon administration to mice in a forced swimming test using animals.

Based on such experimental results, the present inventors have found that the Pleurotus eryngii extract and fractions thereof may be used in a composition for preventing, ameliorating, or treating depression.

Therefore, the present invention may provide a food composition for preventing or ameliorating depression containing an extract or fraction of Pleurotus eryngii as an active ingredient. In addition, it is possible to provide a functional health food for preventing or ameliorating depression, including the food composition.

In the present invention, the extract of Pleurotus eryngii may be obtained by extraction and separation from natural materials using extraction and separation processes known in the art. The extract defined in the present invention may be obtained through extraction from Pleurotus eryngii using an appropriate solvent, and examples thereof may include crude extracts, polar solvent-soluble extracts, and nonpolar solvent-soluble extracts of Pleurotus eryngii.

A solvent suitable for obtaining the extract from Pleurotus eryngii may include water or an organic solvent, and examples thereof may include, but are not limited to, purified water, alcohols having 1 to 4 carbon atoms including methanol, ethanol, propanol, isopropanol, butanol, etc., acetone, ether, benzene, chloroform, ethyl acetate, methylene chloride, hexane, and cyclohexane, which may be used alone or in combination thereof. Preferably, ethanol is used.

The extraction process may include any one selected from among processes such as hot water extraction, cold extraction, reflux cooling extraction, solvent extraction, steam distillation, ultrasonic extraction, elution, and compression. Also, the extract of interest may be additionally subjected to a typical fractionation process or may be purified using a typical purification process.

Pleurotus eryngii suitable for obtaining the extract and fraction may be a Pleurotus eryngii powder obtained by subjecting Pleurotus eryngii to chopping, drying at a temperature of 50 to 70° C. for 10 to 14 hours, and then grinding.

Moreover, the fraction of the Pleurotus eryngii extract according to the present invention is a fraction obtained by performing column chromatography on a solvent extract of Pleurotus eryngii, preferably an ethanol extract obtained using ethanol.

Specifically, the fraction of the Pleurotus eryngii extract may be a primary ethanol fraction (Mixture) obtained in a manner in which the ethanol extract of Pleurotus eryngii is dried to remove ethanol therefrom, dissolved in water, loaded onto an XCD-20 column, washed with PBS buffer, and then eluted using ethanol as an elution solvent.

Also, the fraction of the Pleurotus eryngii extract according to the present invention may be a secondary methanol fraction (R2) obtained in a manner in which the primary ethanol fraction is dried again, dissolved in water, loaded onto an HPLC column, and eluted using a concentration gradient of water and methanol. In an embodiment of the present invention, based on results of analysis of the antidepressant activity of methanol-eluted fractions obtained through HPLC column chromatography, it was confirmed that the R2 fraction had the best antidepressant activity.

Also, the food composition according to the present invention may contain various flavoring agents or natural carbohydrates as additional ingredients like typical food compositions, in addition to the Pleurotus eryngii extract or fraction as an active ingredient.

Examples of the natural carbohydrates may include monosaccharides such as glucose, fructose, and the like; disaccharides such as maltose, sucrose and the like; and polysaccharides such as typical sugars such as dextrin, cyclodextrin, and the like, and sugar alcohols such as xylitol, sorbitol, erythritol, and the like. As the flavoring agents described above, natural flavoring agents (thaumatin), stevia extracts (e.g. rebaudioside A, glycyrrhizin, etc.), and synthetic flavoring agents (saccharin, aspartame, etc.) may be favorably used.

The food composition according to the present invention may be formulated in the same way as a pharmaceutical composition and used as a functional food or added to various foods. Examples of foods to which the composition according to the present invention may be added include beverages, meat, chocolate, foods, confectioneries, pizza, ramen, other noodles, chewing gum, candy, ice cream, alcoholic beverages, vitamin complexes, and health supplements.

Also, the food composition may contain, in addition to the Pleurotus eryngii extract as the active ingredient, various nutrients, vitamins, minerals (electrolytes), flavors such as synthetic flavors and natural flavors, colorants and enhancers (cheese, chocolate, etc.), pectic acid and salts thereof, alginic acid and salts thereof, organic acids, protective colloidal thickeners, pH regulators, stabilizers, preservatives, glycerin, alcohols, carbonating agents used in carbonated beverages, and the like. Furthermore, the food composition according to the present invention may contain fruit flesh for preparing natural fruit juice, fruit juice beverages, and vegetable beverages.

In addition, the present invention provides a functional health food for preventing or ameliorating depression containing the extract or fraction of Pleurotus eryngii as an active ingredient.

The functional health food according to the present invention may be manufactured and processed in the form of tablets, capsules, powders, granules, liquids, pills, and the like.

As used herein, the term “functional health food” refers to food manufactured and processed using raw materials or ingredients having useful functionalities for the human body in accordance with the Functional Health Food Act No. 6727, and means one ingested for the purpose of obtaining useful effects for health such as nutrient control or physiological action on the structure and function of the human body.

The functional health food according to the present invention may contain typical food additives, and the suitability of food additives is determined based on the standards and criteria for the relevant items in accordance with the general rules and general test methods of the Food Additives Code approved by the Food and Drug Administration, unless otherwise specified.

Examples of the items listed in the “Food Additives Code” may include chemical compounds such as ketones, glycine, calcium citrate, nicotinic acid, cinnamic acid, etc.; natural additives such as persimmon color, licorice extract, crystalline cellulose, kaoliang color, guar gum, etc.; and mixed preparations such as sodium L-glutamate preparations, alkaline agents added to noodles, preservative preparations, tar color preparations, etc.

For example, a functional health food in the form of a tablet may be obtained in a manner in which a mixture of the Pleurotus eryngii extract, which is the active ingredient of the present invention, with an excipient, a binder, a disintegrant, and other additives is typically granulated and then compacted using a lubricant, etc. or the mixture is directly compacted. Moreover, the functional health food in the form of a tablet may contain a flavor enhancer and the like, as necessary.

Among functional health foods in the form of capsules, hard capsules may be prepared by filling a typical hard capsule base with a mixture of the Pleurotus eryngii extract, which is the active ingredient of the present invention, with additives such as an excipient, etc., and soft capsules may be prepared by filling a capsule base such as gelatin with a mixture of the Pleurotus eryngii extract with additives such as an excipient, etc. The soft capsules may contain a plasticizer such as glycerin or sorbitol, a colorant, a preservative, and the like, as necessary.

The functional health food in the form of a pill may be formulated by molding a mixture of the active ingredient of the present invention with an excipient, a binder, a disintegrant, etc. using a conventionally known process, and may be coated with white sugar or other coating agents as necessary, or alternatively, the surface thereof may be coated with a material such as starch or talc.

The functional health food in the form of a granule may be prepared by granulating a mixture of the active ingredient of the present invention with an excipient, a binder, a disintegrant, etc. using a conventionally known process, and may contain a flavoring agent, a flavor enhancer, etc. as necessary.

Examples of the functional health food may include beverages, meat, chocolate, foods, confectioneries, pizza, ramen, other noodles, chewing gum, candy, ice cream, alcoholic beverages, vitamin complexes, and health supplements.

In addition, the present invention may provide a pharmaceutical composition for preventing or treating depression containing the extract or fraction of Pleurotus eryngii as an active ingredient.

The pharmaceutical composition according to the present invention is a pharmaceutical composition containing the extract or fraction of Pleurotus eryngii according to the present invention as an active ingredient, and may further contain a pharmaceutically suitable and physiologically acceptable adjuvant in addition to the active ingredient of the present invention. Examples of the adjuvant may include an excipient, a disintegrant, a sweetener, a binder, a coating agent, an expander, a lubricant, a glidant, a flavoring agent, etc.

The pharmaceutical composition is preferably formulated as a pharmaceutical composition by further containing at least one pharmaceutically acceptable carrier in addition to the active ingredient of the present invention for administration.

The pharmaceutical composition may be formulated in forms such as granules, powders, tablets, coated tablets, capsules, suppositories, liquids, syrups, juices, suspensions, emulsions, drops, or injectable solutions. For example, in order to prepare tablet or capsule formulations, the active ingredient may be mixed with an oral non-toxic pharmaceutically acceptable inert carrier such as ethanol, glycerol, water, etc. Also, if desired or required, suitable binders, lubricants, disintegrants, and coloring agents may be incorporated in the mixture. Examples of suitable binders may include, but are not limited to, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride, and the like. Examples of the disintegrants may include, but are not limited to, starch, methyl cellulose, agar, bentonite, xanthan gum, and the like. In compositions formulated as liquid solutions, pharmaceutically acceptable carriers are sterile and biocompatible, and examples thereof may include saline, sterile water, Ringer's solution, buffered saline, albumin injection solution, dextrose solution, maltodextrin solution, glycerol, ethanol, and mixtures of one or more thereof, and other typical additives such as antioxidants, buffers, bacteriostatic agents, etc. may be added as necessary. Also, diluents, dispersants, surfactants, binders, and lubricants may be additionally added, so that injectable formulations such as aqueous solutions, suspensions, emulsions, etc., pills, capsules, granules, or tablets may be formulated. Furthermore, using a method disclosed in Remington's Pharmaceutical Science, Mack Publishing Company, Easton PA as an appropriate method in the field, formulations are preferably prepared depending on diseases or components.

The extract or fraction of Pleurotus eryngii according to the present invention may be contained at a concentration of 1 to 100 μg/ml in the composition of the present invention, and the extract or fraction of Pleurotus eryngii according to the present invention may be contained in an amount of 0.1 to 95 wt % based on the total weight of the composition.

In addition, the present invention may provide a method of preparing a fraction of a Pleurotus eryngii ethanol extract having antidepressant activity.

Specifically, the method includes (1) preparing a Pleurotus eryngii powder by subjecting Pleurotus eryngii to chopping, drying at a temperature of 50 to 70° C. for 10 to 14 hours, and then grinding, (2) obtaining an ethanol extract of Pleurotus eryngii by adding ethanol to the Pleurotus eryngii powder and performing extraction for 20 to 24 hours, (3) obtaining a primary ethanol fraction by subjecting the ethanol extract of Pleurotus eryngii to drying to remove ethanol therefrom, dissolution in water, loading onto an XCD-20 column, and elution using ethanol as a solvent, and (4) obtaining a secondary methanol fraction by subjecting the primary ethanol fraction to drying, dissolution in water, loading onto an HPLC column, and elution using a concentration gradient of water and methanol.

### Mode for Invention

A better understanding of the present invention may be obtained through the following examples. These examples are merely set forth to illustrate the present invention, and are not to be construed as limiting the scope of the present invention.

### Example 1

Preparation of Pleurotus eryngii Extract

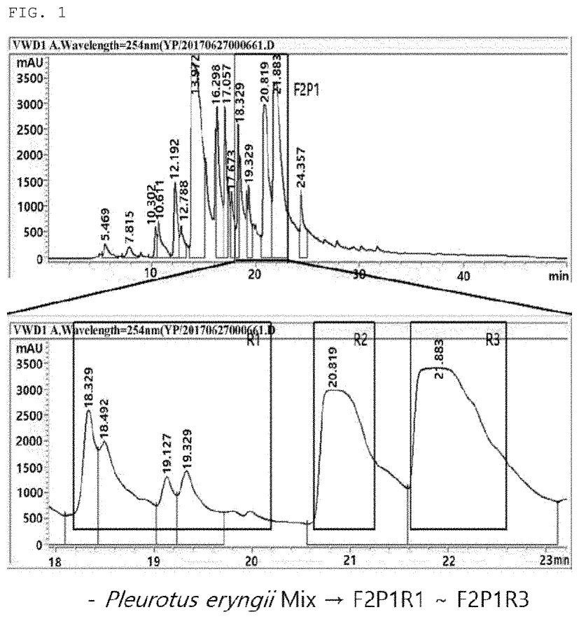

In order to purify fractions having antidepressant activity from Pleurotus eryngii, Pleurotus eryngii was chopped, dried in warm air at 60° C. for 12 hours, and then ground using a grinder to afford a Pleurotus eryngii powder. 500 ml of 99% ethanol (alcohol) was added to 100 g of the Pleurotus eryngii powder, followed by extraction for 24 hours to obtain an ethanol extract. Thereafter, the extract was dried to remove ethanol therefrom, and the dried Pleurotus eryngii extract was dissolved in water, and then a primary fraction (mixture) containing components binding to iron was purified using an XCD-20 column. Here, the resin of the XCD-20 column was equilibrated with PBS buffer for 1 hour, and then the glass column was filled with the resin. Thereafter, the ethanol (alcohol) extract of Pleurotus eryngii was loaded onto the column and then eluted at a rate of 5 ml/min. After completion of elution of the extract, washing was performed with PBS in a volume 10 times the volume of the resin and then materials (primary fraction) attached to the resin were collected using 99% ethanol (alcohol) in a volume 4 times the volume of the resin. Then, the fraction separated by the XCD column was dried, dissolved in water, and then separated again by HPLC to yield a final secondary fraction (R2 fraction) having antidepressant activity from the Pleurotus eryngii extract (FIG. 1). Here, HPLC was performed using a hydrosphere C18 column, and individual fractions were obtained under concentration gradient solvent conditions of water and methanol, which are analysis conditions in the table below, and analysis of activity of the fractions thus separated was performed through experiments of the following examples. Consequently, the highest activity was confirmed in the R2 fraction. The R2 fraction is a fraction eluted in a concentration gradient in which the solvent ratio of water:methanol is 55:45 to 45:55, as a fraction eluted between 19 and 21 minutes.

TABLE 1
HPLC analysis conditions

| Time (min) | % HPLC DW | % MeOH |
|---|---|---|
| 0 | 100 | 0 |
| 1 | 100 | 0 |
| 10 | 90 | 10 |
| 20 | 50 | 50 |
| 40 | 30 | 70 |
| 41 | 30 | 70 |
| 50 | 90 | 10 |

### Example 2

Confirmation of Serotonin Receptor Binding Inhibitory Activity of Pleurotus eryngii Extract Fraction Using Serotonin Receptor Protein

The present inventors analyzed serotonin receptor binding inhibitory activity in order to determine whether the fractions (including the primary fraction and the secondary fraction (R2 fraction)) of the Pleurotus eryngii ethanol extract obtained in Example 1 had antidepressant activity. Whether the fractions of the Pleurotus eryngii extract according to the present invention inhibited binding between a human serotonin receptor and Paroxetin H3 using Paroxetin-H3 with a radioisotope bound thereto, which is a serotonin reuptake inhibitor, was evaluated through radioactivity measurement. Specifically, the human serotonin receptor protein and Paroxetin-H3 were mixed with each of the primary fraction (Mixture) and the secondary fraction (R2) of the Pleurotus eryngii extract at different concentrations (0.01 mg to 1 mg/ml), followed by reaction at 30° C. for 30 minutes. Thereafter, reaction was stopped, and the reaction product was filtered using a GFC filter and washed ten times. After drying the GFC filter well, CPM was measured using a scintillation solution. Here, the standard compound Prozac was used as a positive control.

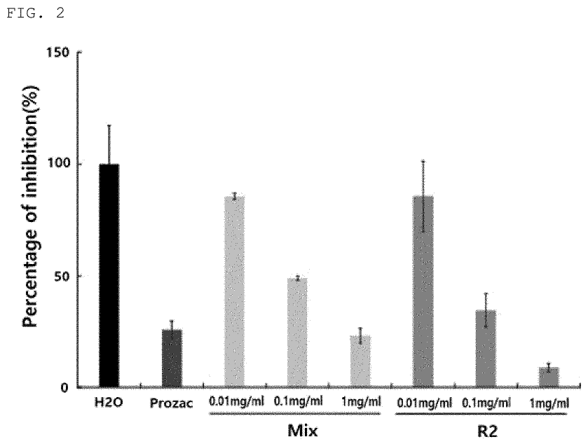

Based on results thereof, as shown in FIG. 2, the Prozac-treated group used as the positive control showed the activity of about 75% on inhibiting binding between the human serotonin receptor protein and Paroxetin-H3. In contrast, the groups treated with 1 mg/ml of the primary fraction (Mixture) and the secondary fraction (R2) of the Pleurotus eryngii extract according to the present invention inhibited binding between the human serotonin receptor protein and Paroxetin-H3 by 78% and 92%, respectively, indicating superior inhibitory activity compared to the positive control.

Thereby, it was found that the fractions of the Pleurotus eryngii extract according to the present invention had the activity of inhibiting binding between serotonin reuptake inhibitors and serotonin receptors, ultimately exhibiting antidepressant activity.

### Example 3

Confirmation of serotonin receptor binding inhibitory Activity after Treatment of Serotonin Receptor-Overexpressing Cell Line with Fraction of Pleurotus eryngii Extract According to the Present Invention

Cancer cell lines are known to overexpress serotonin receptors. Accordingly, the present inventors analyzed whether the fractions of the Pleurotus eryngii extract according to the present invention were able to inhibit binding between a serotonin reuptake inhibitor and a serotonin receptor at the cellular level using an AGS cell line, which is a human gastric cancer cell line.

Specifically, the human AGS cell line was treated with Paroxetin-H3 and each of the primary fraction (Mixture) and the secondary fraction (R2) of the Pleurotus eryngii extract according to the present invention at different concentrations (0.01 mg to 1 mg/ml), followed by reaction for 30 minutes at 30° C. and then washing ten times using a GFC filter. After drying the GFC filter well, CPM was measured using a scintillation solution. Prozac was used as a positive control.

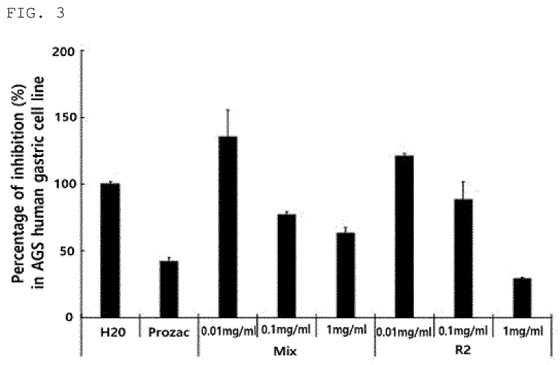

Based on results thereof, as shown in FIG. 3, the Prozac-treated group used as the positive control showed the activity of about 58% on inhibiting binding between the serotonin receptor and Paroxetin-H3 in the AGS cell line. In contrast, when the primary fraction (Mixture) and the secondary fraction (R2) of the Pleurotus eryngii extract according to the present invention were used at concentrations of 1 mg/ml, respective activities of inhibiting binding between the serotonin receptor and Paroxetin-H3 in the AGS cell line were measured to be 38% and 72%. Thereby, it was found that the fractions of the Pleurotus eryngii extract according to the present invention were capable of effectively inhibiting binding between serotonin reuptake inhibitors and receptors thereof by acting on serotonin receptors even in cell lines overexpressing serotonin receptors, as in Example 2.

### Example 4

Confirmation of Signaling Through Binding of Fraction of Pleurotus eryngii Extract According to the Present Invention to Serotonin Receptor

Whether the fractions of the Pleurotus eryngii extract according to the present invention were involved in serotonin receptor-mediated signaling by treating an AGS cell line known to overexpress serotonin receptors with the fractions of the Pleurotus eryngii extract according to the present invention was evaluated using Western blotting. Here, an anti-pERK antibody, anti-Erk antibody, anti-pJNK antibody, anti-JNK antibody, and anti-α Tubulin antibody were used for western blotting.

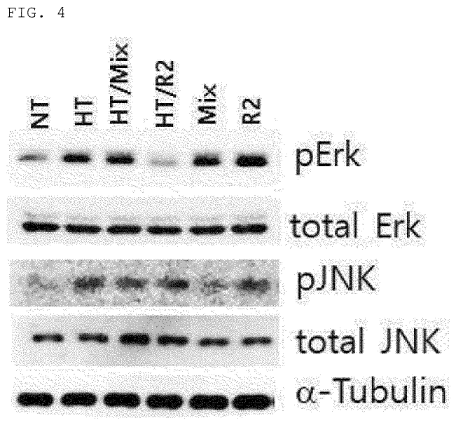

Based on results thereof, as shown in FIG. 4, when the AGS cell line was treated with serotonin, the expression of phosphorylated pErk and pJNK was increased. Also, upon treatment with the fractions of the Pleurotus eryngii extract according to the present invention, the expression of phosphorylated pErk and pJNK was increased as in the serotonin-treated group. Thereby, it was found that the fractions of the Pleurotus eryngii extract according to the present invention activated signaling mediated by serotonin receptors by selectively binding to serotonin receptors, ultimately preventing, ameliorating, or treating depression.

### Example 5

Confirmation of Antidepressant Efficacy of Fraction of Pleurotus eryngii Extract According to the Present Invention Through Forced Swimming Test (FST)

In order to verify whether the fractions of the Pleurotus eryngii extract according to the present invention actually have antidepressant efficacy, a forced swimming test was performed using experimental animals. The antidepressant efficacy was determined by measuring the immobility time during a forced swimming test, and the less the immobility time, the greater the antidepressant efficacy. To this end, mice were intraperitoneally injected with each of the ethanol extract of Pleurotus eryngii according to the present invention and the primary fraction and the secondary fraction of the ethanol extract, after which the immobility time during a forced swimming test was measured. Each sample was injected in an amount of 20 mg/kg into experimental mice. Also, a Prozac-administered group was used as a positive control. Four mice (n=4) were intraperitoneally injected with each sample and then allowed to stand for 30 minutes, after which the mice were submerged in water for 6 minutes and then washed for a while, water in the water tank was exchanged, and the mice were placed again in the water tank for 4 minutes to conduct this experiment. Statistical processing was performed by measuring the immobility time during the 4-minute experimental period.

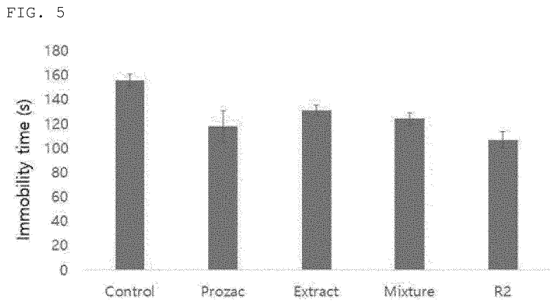

Based on results thereof, as shown in FIG. 5, the Prozac-administered group showed a 24% reduction in immobility time compared to a negative control (depression-induced mice) to which no material was administered. Also, the group administered with the ethanol extract of Pleurotus eryngii according to the present invention showed a reduction in immobility time of about 15%, and the groups administered with the primary fraction (Mixture) and the secondary fraction (R2) showed respective reductions in immobility time of 20% and 32%.

These results indicate that the Pleurotus eryngii extract according to the present invention and the fractions thereof are effective at preventing, ameliorating, and treating depression and are thus usable as antidepressants.

Although preferable exemplary embodiments of the present invention have been disclosed in detail above, it will be obvious to those skilled in the art that the present invention may be implemented in a modified form without departing from the essential characteristics of the present invention. Therefore, the disclosed embodiments are to be considered in an illustrative rather than a restrictive way. The scope of the present invention is indicated in the claims rather than the foregoing description, and all differences within the scope equivalent thereto should be construed as being included in the present invention.

1. A food composition for preventing or ameliorating depression comprising an extract or fraction of Pleurotus eryngii as an active ingredient.

2. The food composition according to claim 1, wherein the extract of Pleurotus eryngii is an ethanol extract of Pleurotus eryngii obtained by adding ethanol to Pleurotus eryngii.

3. The food composition according to claim 1, wherein the fraction of Pleurotus eryngii is a primary ethanol fraction obtained in a manner in which an ethanol extract of Pleurotus eryngii is dried to remove ethanol therefrom, dissolved in water, loaded onto an XCD-20 column, and eluted using ethanol as a solvent; or a secondary methanol fraction obtained in a manner in which the primary ethanol fraction is dried, dissolved in water, loaded onto an HPLC column, and eluted using a concentration gradient of water and methanol.

4. The food composition according to claim 1, wherein the extract or fraction of Pleurotus eryngii inhibits binding between a serotonin reuptake inhibitor and a serotonin receptor, and activates signaling mediated by a serotonin receptor by binding to the serotonin receptor.

5. The food composition according to claim 1, wherein the Pleurotus eryngii is a Pleurotus eryngii powder obtained by subjecting Pleurotus eryngii to chopping, drying at a temperature of 50 to 70° C. for 10 to 14 hours, and then grinding.

6. A pharmaceutical composition for preventing or treating depression comprising an extract or fraction of Pleurotus eryngii as an active ingredient.

7. The pharmaceutical composition according to claim 6, wherein the extract of Pleurotus eryngii is an ethanol extract of Pleurotus eryngii obtained by adding ethanol to Pleurotus eryngii.

8. The pharmaceutical composition according to claim 6, wherein the fraction of Pleurotus eryngii is a primary ethanol fraction obtained in a manner in which an ethanol extract of Pleurotus eryngii is dried to remove ethanol therefrom, dissolved in water, loaded onto an XCD-20 column, and eluted using ethanol as a solvent; or a secondary methanol fraction obtained in a manner in which the primary ethanol fraction is dried, dissolved in water, loaded onto an HPLC column, and eluted using a concentration gradient of water and methanol.

9. The pharmaceutical composition according to claim 6, wherein the extract or fraction of Pleurotus eryngii inhibits binding between a serotonin reuptake inhibitor and a serotonin receptor, and activates signaling mediated by a serotonin receptor by binding to the serotonin receptor.

10. The pharmaceutical composition according to claim 6, wherein the Pleurotus eryngii is a Pleurotus eryngii powder obtained by subjecting Pleurotus eryngii to chopping, drying at a temperature of 50 to 70° C. for 10 to 14 hours, and then grinding.

11. A method of preparing a fraction of a Pleurotus eryngii ethanol extract having antidepressant activity, comprising:
(1) preparing a Pleurotus eryngii powder by subjecting Pleurotus eryngii to chopping, drying at a temperature of 50 to 70° C. for 10 to 14 hours, and then grinding;
(2) obtaining an ethanol extract of Pleurotus eryngii by adding ethanol to the Pleurotus eryngii powder and performing extraction for 20 to 24 hours;
(3) obtaining a primary ethanol fraction by subjecting the ethanol extract of Pleurotus eryngii to drying to remove ethanol therefrom, dissolution in water, loading onto an XCD-20 column, and elution using ethanol as a solvent; and
(4) obtaining a secondary methanol fraction by subjecting the primary ethanol fraction to drying, dissolution in water, loading onto an HPLC column, and elution using a concentration gradient of water and methanol.
